(19) United States
(12) Patent Application Publication
(10) Pub. No.: US 2012/0123287 A1
(43) Pub. Date: May 17, 2012

(54) PORTABLE DEVICE FOR BREATHING DETECTION

(75) Inventor: Andras GEDEON, Stockholm (SE)

(73) Assignees: SEEBREATH AB, Stockholm (SE); MINCOR AB, Stockholm (SE)

(21) Appl. No.: 13/286,442

(22) Filed: Nov. 1, 2011

Related U.S. Application Data
(60) Provisional application No. 61/413,544, filed on Nov. 15, 2010.

(51) Int. Cl. A61B 5/097

(52) U.S. Cl. 600/532

(57) ABSTRACT

A device for detecting breathing of a subject, includes a colorimetric CO2 detector. A method for determining whether a subject is breathing and a method for verifying proper placement of an endotracheal tube when using a resuscitation bag are also described.

### FIELD OF THE INVENTION

The present invention relates to a portable device useful for determining whether a person is breathing. More particularly, the invention relates to a device useful for determining whether a person can breathe on his own, e.g. in connection with Cardio Pulmonary Resuscitation (CPR). Furthermore, the invention relates to a method for determining whether a person is breathing. Moreover the invention relates to a method to verify that an endotracheal tube is correctly placed in the trachea.

### BACKGROUND OF THE INVENTION

There are many situations where there is a need to quickly ascertain if a person is breathing or not. Both in children and in adults the cessation of breathing constitutes an emergency situation where a quick response is of utmost importance. For instance, the chance of a successful resuscitation (CPR) decreases with 7-10% for each minute of delay in initiating CPR.

In a non-hospital setting breathing mainly can be observed through three signs: the movements of the chest, the sounds produced by the air passing through the airways and the feeling of the warm air exhaled.

Chest movements can be irregular and may be difficult to observe reliably in practice. Furthermore they are only very indirect evidence of any actual gas exchanged in the lungs.

Breathing sounds can also be hard to perceive in an emergency situation. Also they can be deceptively weak despite proper breathing as well as very pronounced even when the breathing is minimal when airway obstruction is present.

The feeling of the warmth of the exhaled breath is a more direct measure but it is hard to quantify and particularly in an outdoor setting it can be difficult if not impossible to get a consistent impression.

Sophisticated equipment useful in hospital settings is available for detection of breathing. They use various methods and provide quantitative signals, often based on the observed signs described above. They include sound detection by microphones (U.S. Pat. No. 6,228,037), condensation of water from the warm and humid exhalation on a tip of a fiber (U.S. Pat. No. 5,676,154), sensing of the heat of the exhaled breath by thermistors (U.S. Pat. No. 6,368,287) detection of CO2 in exhaled gas by infrared absorption technology (U.S. Pat. No. 5,095,900) and registration of breathing motion using impedance techniques (U.S. Pat. No. 6,011,477).

Colorimetric carbon dioxide indicating chemistry has been disclosed by an article in Analytical Chemistry Vol 64 page 1383 (1992). These indicators employ pH-sensitive dies and water insoluble organic quaternary (e.g. ammonium or phosphonium) hydroxides, so called phase transfer agents, and are rapidly responding to the changing carbon dioxide concentration.

In order to verify correct endotracheal tube placement, at the start of CPR, colorimetric CO2 sensing elements have been developed (U.S. Pat. No. 4,728,499, U.S. Pat. No. 5,005,572, U.S. Pat. No. 5,179,002 and U.S. Pat. No. 6,502,573). Devices with these elements are temporarily connected to the endotracheal tube immediately after intubation of the unconscious patient to see if the indicator changes color due to the exhaled CO2 when starting to ventilate the patient manually. If so, one may conclude that the tube is correctly placed in the trachea and not, as accidentally happens, in the oesophagus.

In an alternative arrangement, a device with the colorimetric CO2 indicator is attached to the expiratory port of the manual resuscitator (U.S. Pat. Nos. 5,279,289 and 5,679,884). If CO2 is indicated then the tube is properly placed in the trachea.

In both arrangements the device is part of the patient's breathing circuit and therefore suitable for single patient use only. Furthermore, the use is limited to already intubated patients after the start of manual ventilation. Moreover, the units are necessarily bulky since they are designed to be attached to standard respiratory equipment. Finally, the CO2 indicator of the units ceases to function properly after a few hours once taken out of its airtight protective pouch.

A handheld breath detection/confirmation device for multiple use has also been described (U.S. Pat. No. 7,052,470) were an electrically driven breath sensor is combined with an illumination source. This device needs to incorporate a power source both for the light and for the breath sensor and will inevitably be both bulky and expensive.

From the above it appears that there remains a need for a reliable, fast and direct method to establish the breathing status of a person particularly in a non-hospital environment and a device for use in such a method. In order for such a device to be carried by a user at all times, it should be small, lightweight and very simple to use. Prompt and repeated function should be assured for days, if not weeks.

### SUMMARY OF THE INVENTION

The present invention provides a device that preferably meets all the above criteria. The device of the invention is very advantageously compact and adapted for being handheld and is suitable for repeated use, e.g. by emergency medical services in the field. Moreover, during its lifetime, the same device may be used consecutively on many different persons, which is a great advantage over devices that are for single patient use only.

Thus, a device for detecting breathing of a subject is provided, comprising a solid support body having at least one passive part for handling and/or attaching the device and at least one active part; that carries a colorimetric CO2 detector arranged at the at least one active part so as to be exposed to the surrounding air; and suitably comprising a cover for repeatedly separating the CO2 detector from the surrounding air when the device is not in use and allowing the CO2 detector to be exposed to the surrounding air when the device is in use.

The colorimetric CO2 detector(s) suitably is/are arranged on the support body so as to allow said detector(s) to be brought to within a distance of less than 1 cm from the mouth or nostrils of the subject whose breathing is to be detected.

The cover, e.g. a lid or a cover plate, suitable for being repeatedly opened and closed, whereby said cover, when in a closed position separates the colorimetric CO2 detector from the surrounding air, and when in an open position allows the colorimetric CO2 detector to be exposed to the surrounding air.

There also is provided a method for determining whether a subject is breathing comprising positioning the device of the invention in the vicinity of the mouth and/or nostrils of the subject, allowing the colorimetric CO2 detector to be exposed to the air in vicinity of the mouth and/or nostrils of the subject and observing the color of the CO2 detector.

Moreover, a method is provided to determine whether a subject is breathing comprising attaching the device to equipment normally used at the mouth and/or nostrils of a subject, allowing the colorimetric CO2 detector to be exposed to the air in vicinity of the mouth and/or nostrils of the subject and observing the color of the CO2 detector. Examples of such equipment include facemasks, nasal cannulas and pacifiers.

In particular, there is provided a method for verifying proper placement of an endotracheal tube when using a resuscitation bag, said bag comprising an expiratory port, comprising positioning the device according the invention close to the expiratory port of the resuscitation bag, opening the cover of the device so as to allow the CO2 detector to be exposed to air in direct vicinity of the expiratory port and observing the color of the CO2 detector.

### BRIEF DESCRIPTION OF THE DRAWINGS

FIG. 1 is a bottom view of a device of the invention;

FIG. 2 is a top view of the device shown in FIG. 1;

FIG. 3 is a side view of the device shown in FIG. 1;

FIG. 4 is another side view of the device shown in FIG. 1;

FIG. 5 is a top view of a cover plate for separating the CO2 detector from the ambient air,

FIG. 6a is a side view of the cover plate of FIG. 5;

FIG. 6b is another side view of the cover plate of FIG. 5;

FIG. 7 is a top view of a device of the invention with the cover plate of FIG. 5 in an open position;

FIG. 8 is a top view of a device of the invention with the cover plate of FIG. 5 in a closed position;

FIG. 9 is a top view of a seal for protecting the CO2 detector of the invention against air and UV radiation;

FIG. 10 is a top view of a device of the invention before its first use, when the seal is still in place;

FIG. 11 is a side view of the device of FIG. 10;

FIG. 12a-d shows a sandwich structure CO2 detector of the invention, where FIG. 12a is a front view of a plastic film colored frame area surrounding a transparent window area, FIG. 12b is a side view of a sandwich structure consisting of the plastic film of FIG. 12a sandwiched between transparent double-sided adhesive tapes, FIG. 12c is a front view of the CO2 sensing film, FIG. 12d is a front view of a sandwich structure CO2 detector consisting of the sandwich structure of FIG. 12b adhesively attached to the CO2 sensing film of FIG. 12c, and FIG. 12e is a cross sectional view of the CO2 detector of FIG. 12d in place on the support body, in closed position (i.e. when not in use); and

FIGS. 13a-e are side views of different embodiments of the device of the invention.

### DETAILED DESCRIPTION OF THE INVENTION

In a first aspect, a device for detecting breathing of a subject is provided, comprising a solid support body having at least one passive part for handling and/or attaching the device; and at least one active part that carries a colorimetric CO2 detector arranged at the at least one active part so as to be exposed to the surrounding air; and suitably comprising a cover for repeatedly separating each CO2 detector from the surrounding air when the device is not in use and allowing each CO2 detector to be exposed to the surrounding air when the device is use. In one embodiment, the device of the invention comprises one colorimetric CO2 detector. However, it is contemplated that the device also may comprise several colorimetric CO2 detector, of the same or different types.

Very advantageously, the device of the invention comprises a colorimetric CO2 detector arranged on the support body so as to allow said detector to be brought to within a distance of less than e.g. 0.5-5 cm, e.g. less than 0.5-2 cm, or less than 1 cm, from the mouth or nostrils of the subject whose breathing is to be detected.

In one embodiment, the device of the invention comprises a solid support body carrying a CO2 detector and a cover for reducing or minimizing contact between the CO2 detector and the surrounding air when the device is not in use. The cover may be an integral part of the support body or may be separate therefrom. For example, it may be a hollow, tight-fitting space or housing in the support body, wherein the CO2 detector is housed when not in use, or it may be a lid, cover plate or cap, permitting to close off the CO2 detector from the surrounding air.

The cover is suitable for repeatedly permitting the colorimetric CO2 detector to be separated from or exposed to the surrounding air.

The cover may provide for a full or partial enclosure of the CO2 detector. For example, the cover may provide for an airtight seal, i.e. closing off the CO2 detector from the surrounding air in a hermetic fashion. However, it is considered that a less than hermetic separation, simply reducing contact between the ambient atmosphere and the CO2 detector, will generally suffice for the purpose of the invention. Preferably, the cover provides a tight-fitting enclosure for or casing around the CO2 detector.

Before its first use, the device of the invention preferably is provided with a hermetic seal or is enclosed in a hermetic package. This hermetic seal is broken when the device is first taken into use. The cover of the device may be included in the hermetic package, e.g. when it is an integral part of the device or is attached to the device, or may be provided separately.

The solid support body is preferably made of a lightweight material, such as a hard plastic material, e.g. polycarbonate or a styrene-butadiene copolymer, e.g. K-Resin®, from Chevron Phillips Chemical Company LP or a thermoplastic such as Santoprene® (Exxon Mobile). In order to facilitate viewing of the color response of the CO2 detector, the material preferably is transparent to light, at least in that portion of the active part where the CO2 detector is located or in part of the portion where the CO2 detector is located.

Suitably, the support body has a geometrical form and size adapted for being easily transported, e.g. in the breast pocket of a person, allowing it to be rapidly accessible when needed. For example, the device may be of an essentially rectangular shape with a maximum breadth of 1-10 cm, e.g. 3-6 cm, a length of 5-15 cm, e.g. 6-10 cm, and a thickness of e.g. 0.5-10 mm, e.g. 1-5 mm. It should be realized, though, that while a small, easily transported and easy to hold size is generally preferred, the precise geometry and size of the device is not really critical to the functionality of the device and may be varied.

The passive part of the support body essentially is adapted for being held in the grip of the person using the device, e.g. it may have the general form of a handle. The passive part also may comprise means for attaching the device e.g. to the person carrying it, e.g. a clip allowing the device to be attached to a breast pocket; or fastening means for attaching it to a CPR apparatus or any other equipment located in the area of the mouth and/or nostrils of a subject whose breathing is to be assessed. Such equipment may be e.g. a nasal cannula, a face-mask, such as a CPR face mask, or a pacifier.

The active part of the solid support body carries a colorimetric CO2 detector, which is preferably protected against ambient air when not in use, e.g. by means of a cover, lid or cap.

The CO2 detector comprises a porous material containing in its pores a phase transfer agent and a pH sensitive color indicator.

The porous material may be e.g. a porous polymeric material such as known to the person skilled in the art, which material should be capable of absorbing or otherwise binding phase transfer agent and pH sensitive color indicator. In some embodiments, the porous material is a membrane filter, such as a polyethersulfone membrane. However, other porous materials may also be used, e.g. acetylated cellulose layer on a polyester film.

There are a number of pH sensitive dies available to the skilled person, and particular examples are given e.g. in the prior art documents referred to herein above. In one embodiment, the indicator comprises one or several dies selected from thymol blue, cresol red and cresol purple.

The phase transfer agent(s) may comprise at least one water insoluble organic quarternary (e.g. ammonium or phosphonium) hydroxide as described in the prior art documents referred to herein above, e.g. tetraoctyl ammonium hydroxide.

For example, the CO2 detector may comprise a porous material containing in its pores tetraoctylammoniumhydroxide as a phase transfer agent and thymol blue as a pH sensitive color indicator.

The CO2 detector may be attached to the solid support body in a removable or non-removable fashion. For example, the porous substrate of the CO2 detector may be attached to the support body in either a removable or non-removable fashion by means of an adhesive coating or film. In one embodiment, the porous substrate is provided with an adhesive backing permitting it to be releasably attached to the solid support. In this embodiment, when it is desired to replace the CO2 detector, it may be simply removed from the solid support and another one may be attached.

When in use, the CO2 detector responds to the fluctuation in CO2 concentration in the surrounding air by changing color. Thus, on exhalation from an individual, the CO2 detector of the invention, when held at a distance of e.g. 1 cm to 10 cm from the mouth or nose of the individual, will switch from a first color to a second color, due to the increase in ambient CO2. On inhalation, due to the high sensitivity and fast response of the detector, the color of the CO2 detector will switch back to the first hue again, and so on.

The device of the invention may comprise one or more color markings, at least one of which having a color shown by the CO2 detector at a CO2 concentration corresponding to the CO2 concentration of air exhaled from a breathing subject, or the color shown by the CO2 detector at a CO2 concentration corresponding to the “background” CO2 concentration of surrounding air. It may be added that the concentration of CO2 in air exhaled from a breathing subject is about 4% by volume, whereas the concentration of atmospheric CO2, the “background” CO2 concentration, is about 0.04% by volume. Additionally, the device may comprise printed text, e.g. to indicate which color corresponds to exhalation CO2 level.

In some embodiments of the invention, the CO2 detector comprises a plastic film upon which colored areas and optionally text are printed and a multilayered sandwich structure is prepared comprising the plastic film and the CO2 detector. The sandwich structure is attached to the support body by any suitable means.

It is a very advantageous feature of the invention that the device can be functional for more than 24 hours. In order to prolong the lifetime of the CO2 detector further, achieving days or weeks of function, it preferably should be protected from ambient air when not in use. This may be achieved by providing the device with a cover, lid or cap or even by placing the entire device in an airtight container when not in use. It is a preferred aspect of the invention that the device comprises means for rapidly and easily closing off at least the portion thereof which carries the CO2 detector, allowing for rapid opening when the CO2 detector is to be used and easy closure, preferably in between every single use. In one embodiment, this is achieved by the support body comprising a recess or housing wherein the CO2 detector is located and a lid allowing the housing to be opened or closed to the ambient atmosphere. The lid may be a separate unit, such as a cap, or may be a cover or lid attached to the solid body by any suitable means and may be e.g. of the sliding type or hinged or using an adhesive. In one embodiment, the lid, cap or cover is opaque in order to protect the CO2 detector also from UV radiation and light when not in use. The material of the lid, cap or cover may be e.g. a hard plastic material, which also provides mechanical protection of the CO2 detector.

When in a closed position the cover preferably forms an enclosure for the CO2 detector so as to minimize the influx of trace atmospheric gases that may damage the indicator. The cover may form an essentially airtight enclosure for the CO2 detector, although even a less than airtight seal may suffice for the indicated purpose.

In some embodiments, the device comprises a cover plate allowing closing off a space or recess wherein the CO2 detector is located and the support body comprises two parallel slits running parallel on both sides of the recess. The cover plate, adapted for being inserted into the slits, allows for opening or closing the enclosure containing the CO2 detector by gliding in the slits, from an open position, where the cover plate is located towards the passive part of the support body, to a closed position, where the cover plate is located towards the active part of the support body, forming together with the support body an enclosure for the CO2 detector. The open and closed positions of the cover plate may e.g. be defined by a ridge or stop at the bottom of the cover plate and/or the end of the slits. The top of the cover plate may include one or more protruding structures to help the grip of the thumb of the user to push/pull on the cover. The cover may be securely held in open or close position by tight fit in the slits that provides enough friction to prevent inadvertent opening or closing of the device.

In some other embodiments, device comprises a lid that is hinged and allows for exposure of the CO2 detector located in a recess by opening up outwardly. The support body may comprise means for keeping the lid in an open position, or the lid may be kept in an open position by the grip of the hand.

In some embodiments, the support body may have one or more adhesive areas at the edge(s) of the recess housing the CO2 detector and the lid may be held in closed position by releasable attachment to these adhesive areas.

In some embodiments, an adhesive area around the edge of the recess forms a surrounding frame permitting to releasably attach the cover plate. In this case, the support body may comprise, e.g. at the proximal side of the recess, a slit for the cover plate. The cover plate comprises a ridge at one or both ends, functioning both as a handle for the cover plate and to stop the cover plate from falling through the slit.

In some embodiments, the cover is a cap suitable for enclosing the active part of the support body. The cap may be anchored to the support body, e.g. by a wire, a coil spring, an adhesive strip or a cord, in order to prevent it from being lost when not in place.

In some embodiments, the support body comprises a hollow housing with the orifice at the distal end, and the CO2 detector is mounted on a movable part inside the housing that can be easily manoeuvred by the user, so as to extend the CO2 detector out of the housing when in use and withdrawing it again back into the housing after use. The orifice of the housing may be closed off by a cap or the geometry of the movable part inside the housing may be such as to close off the CO2 detector when inside the housing; for example the movable part may comprise a tight-fitting bottom plate at the active part. The housing preferably is tight-fitting around the CO2 detector-movable part assembly.

In some embodiments, the CO2 detector is a multilayered (sandwich) structure. The structure comprises a transparent plastic film printed with at least one reference color in part of the film area, e.g. in the peripheral area of the film. For example, the plastic film may be printed around the whole periphery so as to provide a frame area having a reference color surrounding a transparent window area, or may be printed in at least in part of the periphery. In one embodiment, the plastic film is printed in one area, e.g. at one border, with one reference color (e.g. color corresponding to exhalation CO2 level) and in another area, e.g. at an opposite border, with the other reference color (i.e. color corresponding to atmospheric CO2 level).

The plastic film also may comprise printed text, e.g. to indicate which color corresponds to exhalation CO2 level and which color does not although, as pointed out herein above, such information also may be printed directly on the support body.

The plastic film is sandwiched between two transparent double-sided adhesive tapes, i.e. a bottom tape and a top tape, chosen so as not to interfere with the chemical function of the CO2 indicator. The tapes may be e.g. of polypropylene, covered by an adhesive, e.g. chosen from the synthetic rubber family. For example, the tape may be a double sided filmic tape of the synthetic rubber type. A CO2 sensing film of suitable size and form is attached to the top tape of the sandwich structure. The CO2 sensing film is of a porous material, such as a membrane filter of the polyethersulfone membrane type. The CO2 sensing film is impregnated with the indicator, comprising e.g. tetraoctylammoniumhydroxide as a phase-transfer agent, with thymol blue as a pH-sensitive color indicator.

The size and form of the CO2 sensing film as well as the location of attachment of the film on the top tape are such as to allow the reference color area of the plastic film to be observed from both sides of the sandwich structure. In case the printed reference color area of the plastic film forms a frame, the size and form of the CO2 sensing film suitably are such as to correspond to the window area of the plastic film and the CO2 sensing film then is attached to the top tape at a location corresponding to the window area of the plastic film. By means of the bottom tape, the sandwich structure CO2 detector is attached to the support body. At least in the area of attachment of the CO2 detector the support body the material of the support body preferably is transparent to light, allowing the color of the CO2 detector to be observed from both sides of the device.

In some embodiments, the CO2 detector comprises a reference-colored frame of plastic film sandwiched between transparent double-sided adhesive tapes and a CO2 sensing film adhered at one surface of the sandwich structured frame, the other surface of said frame serving to attach the CO2 detector to the support body.

Many variants of the design of the device of the invention may be conceived by a person of ordinary skill in the art and the invention is by no means limited to those that are described and exemplified herein.

In a situation where CPR is performed, the device of the invention suitably is held adjacent to the expiratory port of a manual resuscitator or is temporarily attached close to the port. In this way the device can also be used to verify proper endotracheal tube placement when CPR has been initiated.

Very advantageously, the device of the invention may be adapted for being attached to any equipment located in the area of the mouth or nose of a subject, in particular equipment for individuals that are in need of respiratory help or whose breathing is to be assessed. Thus, the device may be attached to a nasal cannula, a face-mask, such as a CPR face mask, or to a pacifier.

There also is provided a method for detecting breathing of a subject, comprising holding the device according to the invention in the vicinity of the mouth and/or nostrils of the subject and observing the color of the CO2 detector. The subject preferably is a human, but the method equally well may be performed on any mammal, such as a dog, a cat, or a horse.

By the expression “vicinity of the mouth and/or nostrils” as used herein, is meant at a distance of e.g. 0.5 cm to 10 cm, or 1 cm to 5 cm. It should be realized that the distance at which the CO2 detector is suitably held varies and may depend on factors such as the surroundings and the strength with which the person is breathing. For example, in a perhaps windy outdoors environment and/or with a person having a very shallow breath the device may have to be held at a very close distance of the nostrils or the mouth in order to give a reliable signal.

The colorimetric CO2 detector, due to its fast response, will change color during a breath cycle of the subject the breathing of which is to be determined. For example, in some embodiments, the CO2 detector has a first color at the end of the inhalation phase of the breathing cycle and on exhalation from the subject swiftly changes its color to a second color, and so on. The person using the device will normally be able to reliably determine whether the subject is breathing after an observation period of as short as a few seconds.

According to one aspect of the invention, there is provided a method for verifying proper placement of an endotracheal tube during intubation, e.g. when using a resuscitation bag. A resuscitation bag may be e.g. a manually operated self-inflating resuscitation bag, or a flow-inflating or gas-powered resuscitation bag. The resuscitation bag comprises a patient connection port, to which the endotracheal tube can be connected and an expiratory port through which air exhaled from the patient exits.

In order to verify proper placement of the endotracheal tube used to intubate a subject in need thereof, the device according the invention is positioned close to the expiratory port of the resuscitation bag, the CO2 detector is exposed to air in direct vicinity of the expiratory port and the color of the CO2 detector is observed. Exposure of the CO2 detector is achieved by opening the cover separating the CO2 detector from surrounding air, either before or after positioning the device of the invention close to the expiratory port.

The device of the invention may be simply held by hand close to the expiratory port, or may be attached to the resuscitation apparatus, in close vicinity of the expiratory port, e.g. using fastening means provided at the passive part of the device.

### EXAMPLES

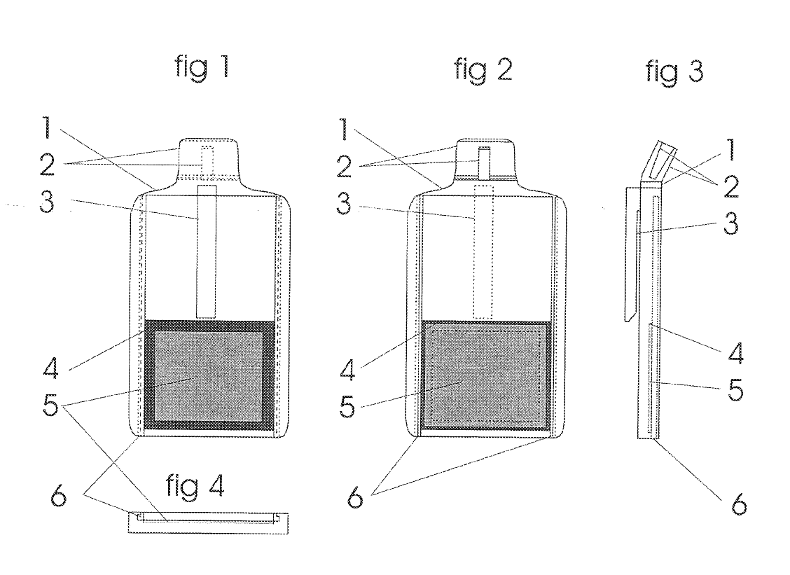

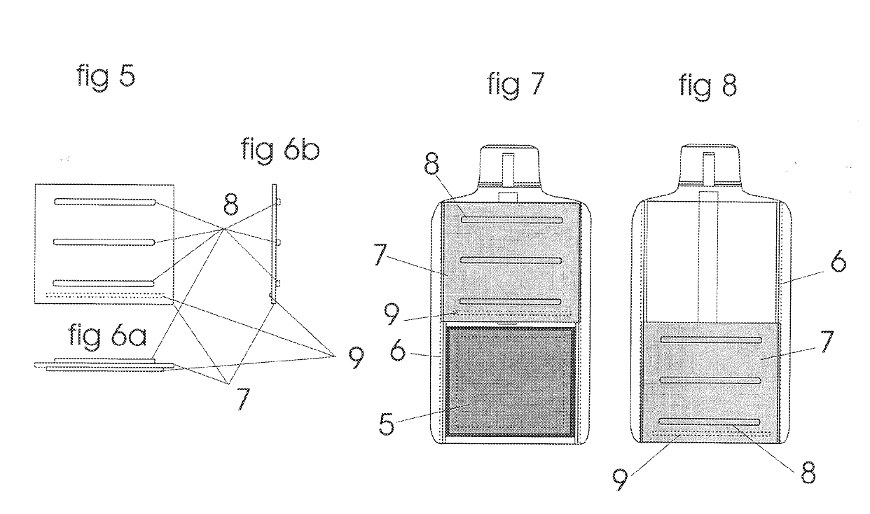

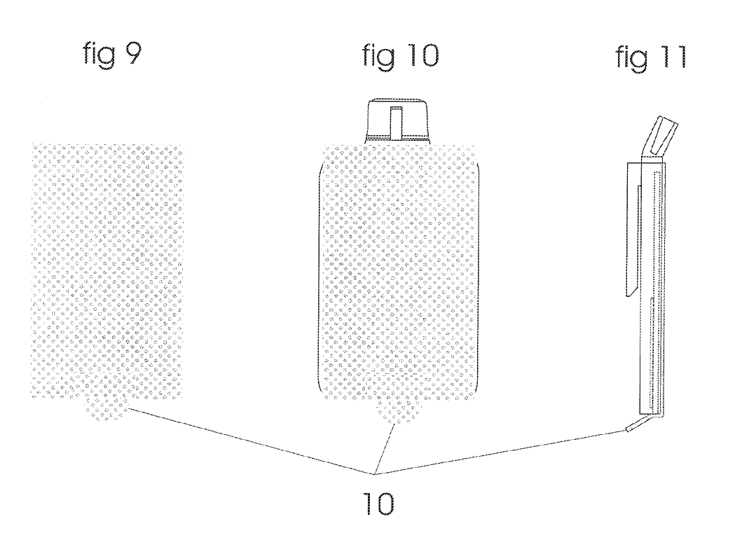

FIGS. 1-13 illustrate some preferred embodiments of the invention.

FIGS. 1-8 show a device of the invention at an approximately 1:1 scale. The support body 1 is made of a transparent plastic material such as polycarbonate or K-resin and is basically a thin plate. One end, the active part, of the support body 1 carries a CO2 detector 5 while the other, passive part of the support body 1 is intended for the grip of the user or for attachment of the device to e.g. a CPR apparatus.

The passive part of the support body, serving mainly as a handle for the user to hold, is split (FIG. 3) providing a grip 2 to facilitate the temporal attachment of the device to other devices, such as a CPR apparatus. The back side of the support body incorporates a clip 3 to keep the device in place in between uses, for instance in a breast pocket, or to allow attaching the device in any other suitable way to the user.

The active part of the support body 1 carries CO2 detector 5, which includes an integrated reference color area 4 to help facilitate perception of a color change. Since the support body 1 is transparent to light, the CO2 detector 5 may be observed not only from the front (FIG. 2), but also from the back (FIG. 1), which facilitates observation of the color of the CO2 detector 5 when the device is held against the mouth and/or nostrils of a subject, the breathing of which is to be determined.

The support body 1 is adapted to receive a cover plate for protecting the CO2 detector 5 against ambient air in between use. Thus, two slits 6 are provided, running parallel along the long edges of the support body 1, at both sides of the CO2 detector 5. A cover plate 7 (FIGS. 5, 6a and 6b) can be inserted into slits 6 and will be able to glide along them between an open position (FIG. 7), exposing the CO2 detector 5 to ambient air, and a closed position (FIG. 8), separating the CO2 detector 5 from ambient air. The top of the cover plate 7 is fitted with protruding structures 8 to help the grip of the thumb of the user to push/pull on the cover between the open and closed positions. A protrusion 9 at the bottom of the active part of the cover plate 7 and/or the active part of the slits determine the appropriate positions as shown in FIGS. 7, 8 and 12e.

When stored prior to use, the CO2 detector is sealed off in an airtight way from the ambient atmosphere and protected from unwanted UV-radiation by a seal 10 (FIG. 9) that is attached to the top and the front surface of the device (FIGS. 10 and 11). The seal is made of a standard laminate of aluminium and polyethylene and is removed and discarded when the device is taken into use.

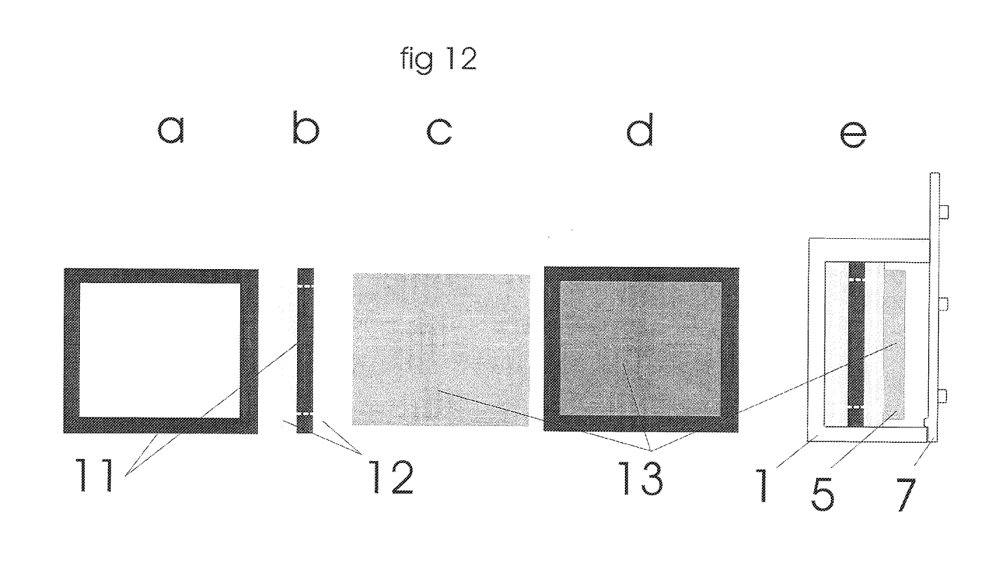

In the illustrated embodiment, the CO2 detector 5 is a multilayered structure as explained with reference to FIG. 12. The structure comprises an adhesive frame 11 punched from a multilayer structure comprising a plastic film (FIG. 12a), in a reference color, sandwiched between two identical transparent double adhesive tapes 12, as shown in FIG. 12b. The carrier of the tapes 12 is of polypropylene and the adhesive is chosen so as not to interfere with the chemical function of the CO2 sensing film 13. Suitable adhesives are chosen from the synthetic rubber family. The CO2 sensing film 13 (FIG. 12c) of suitable size is attached to the adhesive frame (FIG. 12d).

The CO2 sensing film 13 is of a porous material such as a membrane filter and contains the active chemical, e.g. tetraoctylammoniumhydroxide with thymol-blue as a pH-color indicator. The CO2 detector 5 is attached to the support body 1 (only schematically shown in FIG. 12e) and may be separated from the ambient atmosphere using a cover plate 7 as shown in FIG. 12e.

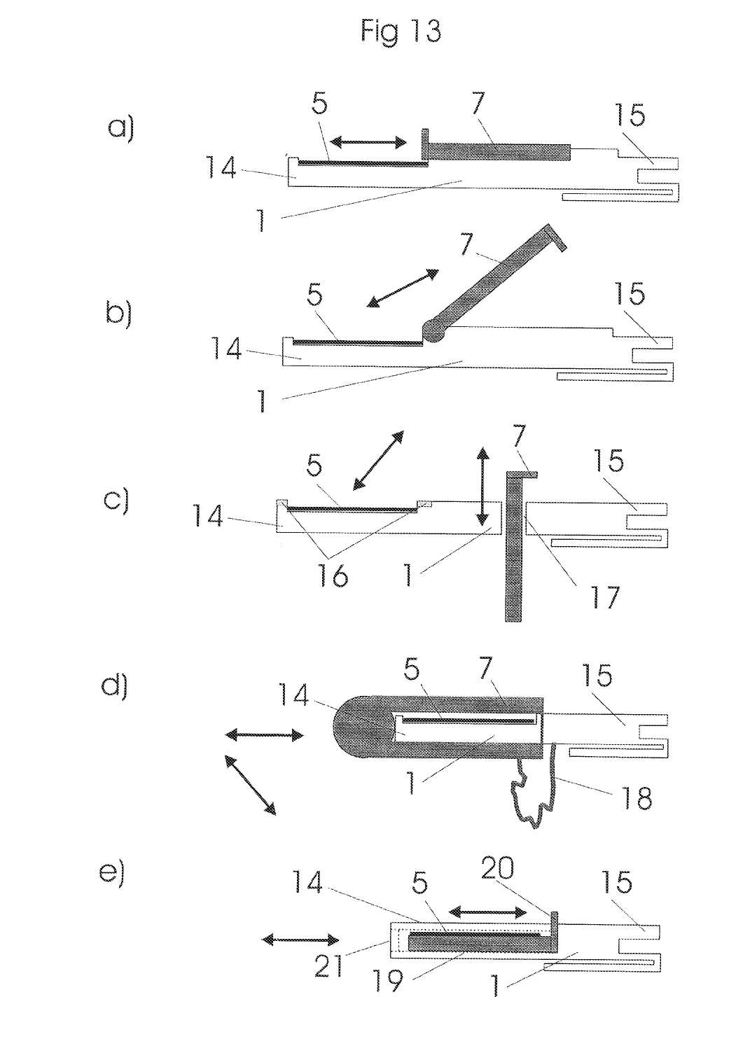

FIGS. 13a-e show some further embodiments of the invention. The support body 1 has an active part 14 where the CO2 detector 5 is located and a passive part 15 where it is held by the user. The passive part 15 is configured to allow for temporal attachment of the device to the user's clothing or other places where it is conveniently kept. A protective cover 7 is provided that can be easily manoeuvred to either close off the CO2 detector 5 from the ambient air or to expose it to the ambient air. FIG. 13a shows the preferred embodiment with a sliding cover 7. In FIG. 13b the cover 7 is hinged but performs the same function. In FIG. 13c the cover 7 is attached to or detached from an adhesive frame 16 that is provided around the CO2 detector 5. Also shown is that, when detached from the adhesive frame 16, the cover 7 rests in a slit 17 in the support body 1, easily at hand. FIG. 13d shows a cover 7 in the form of a cap that is placed around the end of the active part 14 of the support body 1 and is attached flexibly, e.g. with a cord 18, to the passive part 15 of support body 1.

Finally, in FIG. 13e the support body 1 comprises a hollow space or housing 19 and the CO2 detector 5 is mounted on a movable part 20, allowing it to be withdrawn into the housing 19 when not in use and pushed out of the housing 19 so as to extend out of the orifice 21 of the housing 19 when in use. In this embodiment, the device optionally may include a cap (not shown), which may be placed onto the active part 14 of the support body 1 e.g. during longer periods of non-use.

1. A device for detecting breathing of a subject, comprising a solid support body having
(i) a passive part for handling and/or attachment; and
(ii) an active part that carries a colorimetric CO2 detector;
said detector being arranged on the support body so as to be exposed to the ambient air when the device is in use.

2. The device of claim 1, wherein the CO2 detector is arranged on the support body so as to allow said detector to be brought to within a distance of less than 1 cm from the mouth or nostrils of the subject whose breathing is to be detected.

3. The device of claim 1, comprising a cover for separating the CO2 detector from ambient air when the detector is not in use.

4. The device of claim 3, wherein said cover is a lid, a cap or a housing that can be used repeatedly to separate the CO2 detector from ambient air.

5. The device of claim 3, wherein said cover, when in a closed position, provides an airtight enclosure for the CO2 detector.

6. The device of claim 1, wherein the CO2 detector comprises a porous material containing in its pores a phase transfer agent and a pH sensitive color indicator.

7. The device of claim 6, wherein the phase transfer agent is tetraoctylammoniumhydroxide and the pH sensitive color indicator is thymol blue.

8. The device of claim 1, wherein the support body at least in part is made of a material transparent to light.

9. The device of claim 1, comprising one or more colored areas, at least one colored area having a color shown by the CO2 detector at a CO2 concentration corresponding to the CO2 concentration of air expired from a breathing subject or a color shown by the CO2 detector at a CO2 concentration corresponding to the CO2 concentration of surrounding air.

10. A method for determining whether a subject is breathing comprising positioning the device according to claim 1 in the vicinity of the mouth and/or nostrils of the subject, allowing the CO2 detector to be exposed to air in vicinity of the mouth and/or nostrils of the subject and observing the color of the CO2 detector.

12. The method according to claim 10, comprising attaching the device to equipment located at the mouth and/or nostrils of the subject, such as a pacifier, a nasal cannula, or a facemask.

13. A method for verifying proper placement of an endotracheal tube when using a resuscitation bag, said bag comprising an expiratory port, comprising holding the device according to claim 1 close to the expiratory port of the resuscitation bag, and observing the color of the CO2 detector.
